(19) United States
(12) Patent Application Publication
(10) Pub. No.: US 2017/0059541 A1
(43) Pub. Date: Mar. 2, 2017

(54) Outgas Measurement Device

(71) Applicant: Gregory Howard, Reno, NV (US)

(72) Inventor: Gregory Howard, Reno, NV (US)

(73) Assignee: TrenLot, Inc., Reno, NV (US)

(21) Appl. No.: 15/254,936

(22) Filed: Sep. 1, 2016

Related U.S. Application Data
(60) Provisional application No. 62/212,769, filed on Sep. 1, 2015.

(51) Int. Cl. G01N 33/00; G01N 33/14; G01N 27/04

(52) U.S. Cl. CPC G01N 33/0042; G01N 27/04; G01N 33/0047; G01N 33/0037; G01N 33/146

(57) ABSTRACT

An apparatus is described to detect, measure or both, key compounds, such as sulfur dioxide (SO2) associated withoutgassed elemental compounds suggestive of the composition of one or more constituents in a liquid.

### CROSS-REFERENCE TO RELATED APPLICATIONS

The present application for patent claims priority through the applicant's prior provisional patent application, entitled:
1. Outgas Measurement Device, Ser. No. 62/212,769, filed Sep. 1, 2015; which provisional application is hereby incorporated by reference in its entirety.

### COPYRIGHT

A portion of the disclosure of this patent document contains or may contain material subject to copyright protection. The copyright owner has no objection to the photocopy reproduction of the patent document or the patent disclosure in exactly the form it appears in the Patent and Trademark Office patent file or records, but otherwise reserves all copyright rights.

### FIELD OF TECHNOLOGY

The technology of the present application relates to an electrochemical sensor apparatus, and more particularly an electrochemical sensor apparatus configured to determine constituents, such as, for example, SO2 presence and levels, in barrels and tanks made of materials such as, for example, stainless steel and wood.

### BACKGROUND

It is widely known that elemental sulfur dioxide (SO2) has played a role in preserving wine. It has also acted as an anti-microbial agent and antioxidant that curtailed the growth of undesirable bacteria and yeasts. Without appropriate or near-appropriate levels of SO2, wine typically degraded in quality and spoiled. Aging of whiskey and wine, for example, traditionally occurred in a wood or stainless steel barrel or tank. Historically, in the absence of usable liquid based sensors, measurements in wine barrels involved, opening the barrel or tank, removing a sample of the wine, and testing the wine in a reagent based process to determine the level of free SO2 dissolved in the liquid.

Conventional SO2 titration testing methods have all involved removing a sample and adding reagents that contaminate the wine. Optical methods for determining SO2 levels and other chemical properties in liquids have also included removing a sample. Further, equipment associated with such methods have been costly, often making it cost prohibitive to constantly measure important properties, such as SO2.

As a result, expensive sample based SO2 testing has generally forced wine makers to use random sampling methods to test a subset of the total barrels for SO2 levels. For example, wine industry standard practices dictate random sampling of roughly 5% to 10% of all barrels once per month.

Distillery industry practices generally have not included a sample based reagent or optical test. Rather, a master distiller (or equivalent) will open one whisky barrel (also sometimes referred to as a cask) and taste the whisky to determine the quality. Distilleries have found it difficult to determine what barrels produce high quality whisky because a significant part of the whisky quality is determined by the quality of the barrel.

Brewery industry practices typically included aging beer in oak barrels. Industry practices for preservatives, such as, for example, SO2, vary and have included other chemicals, such as potassium sorbate, sorbic acid, sodium sorbate, caldum sorbate, and others. Breweries have often used CO2 gas as a preservative and as a way to insulate beer aging in an wood barrel from oxygen. In addition, some wood barrels were pressurized with CO2 in order to keep aging beer from spoiling.

### BRIEF SUMMARY OF SOME ASPECTS OF THE DISCLOSURE

The applicants believe that they have discovered at least one or more of the problems and issues with systems and methods noted above as well as advantages variously provided by differing embodiments of the outgas measurement device disclosed in this specification. In some embodiments, the outgas measurement device includes at least the combination of a hollow housing, a porous membrane, and a gas measurement sensor, such as an electrochemical gas measurement sensor. The device can measure the outgassed property of a specific constituent, such as SO2 levels, indicating the level of SO2 in the liquid, such as, for example, wine. In some instances, measuring outgassing properties, such as SO2, can result in obtaining a projected SO2 level in the liquid on an ongoing basis. Further, obtaining such measurements can occur without sampling, and thus, without opening the container holding the liquid.

Using electrochemical sensors to monitor the properties of outgasses compounds, such as those naturally occurring from the decomposition of wood barrel material, can provide information suggestive of the quality of a whisky barrel. Outgassed compounds may include hydrocarbon based gases such as Methane (CH4). In addition, overall combination of constituents, such as outgassed ethanol alcohol (C2H5OH), hydrocarbons, and nitrogen dioxide (NO2) may provide a measure of projected quality. Using an electrochemical sensor to measure the amount of outgassed SO2 can be correlated for gross estimation of the total parts per million (ppm) of free SO2 in the wine.

The foregoing has outlined rather broadly the features and technical advantages of examples. The conception and specific examples disclosed may be readily utilized as a basis for modifying or designing other structures for carrying out the same purposes of the present disclosure. Features which are believed to be characteristic of the concepts disclosed herein, both as to their organization and method of operation, together with associated advantages will be better understood when considered in connection with the accompanying figures. Each of the figures is provided for the purpose of illustration and description only, and not as a definition of the limits of the claims included herein now or as amended during prosecution. Thus, the scope of a given claim is to be determined by the claim as issued and not by whether it addresses an issue set forth in the above Background or includes a feature set forth in this Brief Summary.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a drawing, in perspective, of an exploded view of an outgas measurement device; and

FIG. 2 is a drawing, in perspective, of an arrangement of the components of the outgas measurement device of FIG. 1.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION OF THE PREFERRED AND OTHER EMBODIMENTS

The following description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

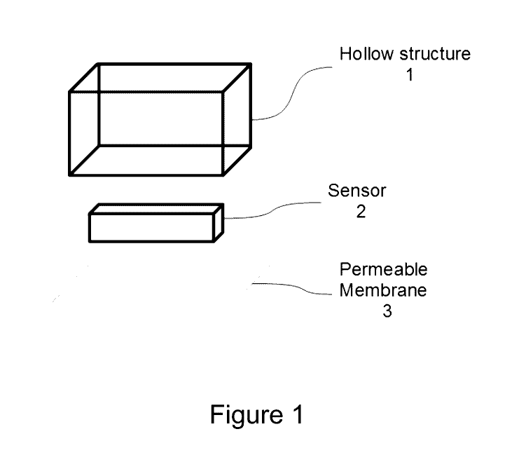

Referring now to FIG. 1, in some embodiments, a hollow housing 1 can be constructed from a food grade material, such as, for example, high density polyethylene (HDPE), low density polyethylene (LDPE), or Polypropylene. The sensor 2 is a gas sensor, such as, for example, an electrochemical gas sensor, used to measure one or more specific constituents, such as sulfur dioxide (SO2). A semi-permeable membrane 3 can keep the gas sensor free from moisture. The semi-permeable membrane, in some instances, is made up of a food grade porous material such as polytetrafluoroethylene (PTFE), also known as Teflon, Fluon, Hostaflon, and Polyflon. PTFE materials with various pore sizes are readily available from vendors such as St Gobain, Zeus, Inc. and others. The pore size is small enough to keep fluid out, such as wine, and let gas compounds in, such as sulfur dioxide (SO2), through the membrane to the electrochemical sensor.

Still referring to FIG. 1, in some embodiments, the outgas measurement device includes a hollow structure 1 which can serve as a housing with one end securely covered with a semi-permeable membrane. One or more sensors 2 can, in whole or in part, be positioned within the hollow portion of the housing 1. In some instances, the combination of a hollow structure 1 and semi-permeable membrane 3 create a fluid-resistant sensor housing providing the sensor 2 to be submerged in or sit adjacent to one or more fluids being measured, such as, for example, wine or whisky. The hollow structure 1, and semi-permeable membrane 3, can include materials resistant to decay or degradation as a result of contacting the fluid, such as wine, being measured. The sensor 2, housing 1, and semi-permeable membrane 3, can be constructed from materials that can come into contact with food, specifically wine and whisky. In some instances, food grade silicon barrel bungs produce an airtight seal around the barrel bung hole.

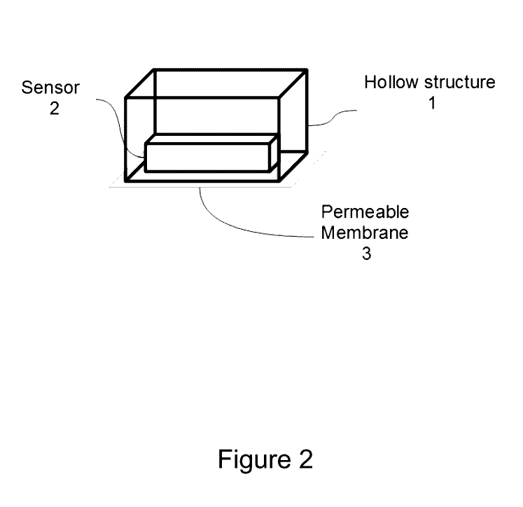

Referring now to FIG. 2, in some embodiments, the sensor 2 is positioned internal to the hollow structure 1, with the sensing portion of the sensor 2 proximal to the semi-permeable membrane 3. The semi-permeable membrane 3 can cover an open end of the hollow structure, such open end proximal to the liquid. In some embodiments, this device can be part of a barrel enclosure, the device being inserted into the barrel bung hole in, for example, a wine or whisky barrel.

In some embodiments, the sensor 2, is connected to a data collection mechanism, such as, for example, an mechanism including one or more of a receiver, a processor, a memory, and a transmitter. The data collection mechanism can collect, process, transmit, and/or store data obtained from the sensor 2. In some implementations, an electrochemical SO2 sensors is used, such as the model number ME-3-S02 from Zhengzhou Winsen Electronics Technology Co., Ltd. and the model number 4SO2-20 from Shenzhen Keep Power Electronics Co., Ltd. In some instances, the sensor is connected by attaching control and voltage wires from a PCB board with appropriate sensor control components. In some instances, electrochemical sensor vendors can provide the PCB with necessary electronic components to operate the sensor. The sensor output can be a voltage that can be measured with a multi meter or similar component on a PCB. In some instances, the SO2 electrochemical sensor includes one or more of a supply voltage, a reference voltage, and a measurement voltage. The reference voltage can be subtracted from the measurement voltage after the sensor has warmed up, such as for a thirty second warm-up period.

Electrochemical sensors typically operate by reacting with a gas, such as SO2, that produces an electrical signal proportional to the gas concentration. The electrochemical sensor can include a sensing electrode (also known as a working electrode) and a counter electrode separated by a layer of electrolyte. In some instances, SO2 particles, for example, that contact the sensor pass through a small capillaries that diffuse SO2 through the hydrophobic barrier before they reach the electrode. Gas contacting the electrode produces a measurable electrical signal with a resistor connected across the electrodes. In some sensor implementations, the current is equal to the gas concentration that is flowing between the anode and the cathode.

Once the hollow structure 1, is suitably fastened to the semi-permeable membrane, such as with a mechanical clamp or food grade hot or cold glue, the sensor can be placed in the housing. In some SO2 measuring embodiments, the porous food grade membrane is made of PTFE with a pore size of 5 to 10 microns. Pore size is selected based on the properties of the liquid and the gas being measured. In some embodiments, pore size is large enough to let elemental SO2 partials through the membrane in quantities large enough to be detected by the electrochemical sensor. In some instances, PTFE material with pore sizes between 5 to 10 microns is used. The pore size can prevent liquid, such as wine, from entering the hollow structure 1 while allowing constituent gas or gases to pass into the hollow structure.

In some embodiments the hollow structure 1, may vary in size and shape. Shape of the hollow structure 1, may change to fit an enclosure orifice. In some embodiments, the hollow structure 1, size and shape may be changed to accommodate one or more sensors. In one or more embodiments, a pressure sensor added to the hollow structure may be present proximal to the hollow structure with one or more semi permeable membranes. A combination of one or more sensors may receive compounds for detection passing through one or more membranes having one or more pore sizes with one or more pore size ranges in order to, in some instances, filter constituents for detection. In some embodiments, one or more sensors may benefit from one or more semi permeable membranes. In some embodiments one or more semi permeable membranes in one or more combinations may include, for example pore sizes that range from 0 to 5 microns, 5 to 10 microns, 10 to 15 microns, 15 to 20 microns, 20 to 25 microns, 25 to 30 microns, 30 to 35 microns, 35 to 40 microns, 40 to 45 microns, 45 to 50 microns, and 0 to 50 microns.

The amount of SO2 detected by the electrochemical sensor may not be the same as the amount of SO2 present in the wine. However, measured proportionate values of outgassed SO2 can be used to determine the amount of free SO2 present in the wine. Estimated SO2 levels used to detect free SO2 levels can at least in part, be based on the outgassed SO2 measurement. Implementing the outgas measurement device with barrels of aging wine can provide a monitoring system that detects imbalances of SO2.

Further analysis of gas based SO2 measurements through the outgas measurement device can be transmitted from the outgas measurement device to a computer or data logger for further trending and analysis. Each barrel can be fundamentally different based, at least in part, on the barrel or receptacle material. Organic material, such as oak wood, can vary in density and porosity, creating a different aging environment for wine, whisky, or other substance. The collection and analysis of sensor data provided by the outgas measurement device can be further correlated with additional environmental conditions, such as humidity and temperature. Outgassing rates of materials such as SO2 can vary with temperature and pressure and can be taken into consideration when using the outgas measurement device sensor data to estimate the rough level of SO2 in the liquid, such as wine.

In some cases, wine slowly evaporates through a wood barrel, reducing the total amount of wine within a barrel and allowing a headspace (air gap) to exist. During the aging process of wine, oxygen can be detrimental to the wine, causing oxidation and providing fuel for macrobiotic organisms to potentially affect the wine. Locating the outgas measurement device in, for example, the barrel bung, and encased in a material such as, for example, food grade silicon, can provide a way to routinely wash or rinse the semi-permeable membrane material during normal wine production practices, such practices including, for example, each barrel being opened and “topped off” with wine to reduce or remove certain gasses, such as oxygen, from the barrel headspace.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

What is claimed is:

1. An outgas measurement device, comprising:
a housing wherein the housing is hollow;
a porous membrane; and
a measurement sensor, wherein the measurement sensor is an electrochemical gas measurement sensor.

2. The outgas measurement device of claim 1, wherein the measurement sensor measures outgassed constituents in one or more barrels.

3. The outgas measurement device of claim 2, wherein the one or more barrels comprise one or more of a wine barrel, a whisky barrel, or a beer oak barrel.

4. The outgas measurement device of claim 1, wherein the measurement sensor measures outgassed constituents in one or more tanks.

5. The outgas measurement device of claim 4, wherein the one or more tanks comprise one or more of a wine tank, or a whisky tank.

6. The device of claim 1, wherein the measurement sensor measures sulfur dioxide.

7. The device of claim 1, wherein the measurement sensor measures one or more outgasses compounds naturally occurring from the decomposition of wood barrel material.

8. The device of claim 7, wherein the one or more outgasses compounds comprise one or more hydrocarbon based gases.

9. The device of claim 8, wherein the one or more hydrocarbon based gases comprise Methane (CH4).

10. The device of claim 1, wherein the measurement sensor measures one or more of outgassed ethanol alcohol, hydrocarbons, and nitrogen dioxide.
